(19) United States
(12) Patent Application Publication
(10) Pub. No.: US 2024/0103000 A1
(43) Pub. Date: Mar. 28, 2024

(54) AUTOMATIC DEVICE FOR NON-INVASIVE MALARIA DIAGNOSIS THROUGH OPTICAL REFLECTANCE TECHNIQUES, METHODS AND USES THEREOF

(71) Applicant: Universidade Do Minho, Braga (PT)

(72) Inventors: Susana OLIVEIRA CATARINO, Braga (PT); Vitória DA CUNHA BAPTISTA, Ponte De Lima (PT); Maria Isabel MENDES VEIGA, Guimarães (PT); Graça Maria HENRIQUES MINAS, Braga (PT)

(21) Appl. No.: 18/246,760

(22) PCT Filed: Sep. 29, 2021
(86) PCT No.: PCT/IB2021/058926

§ 371 Date: Mar. 27, 2023

(30) Foreign Application Priority Data
Sep. 29, 2020 (PT) 116798

(51) Int. Cl. G01N 33/569; A61B 5/00; B01L 3/00; G01N 33/72

(52) U.S. Cl. CPC G01N 33/56905; A61B 5/0075; B01L 3/502715; G01N 33/72; A61B 2560/0223; B01L 2200/18; B01L 2300/0654; G01N 2333/445

(57) ABSTRACT

A portable device for detecting and/or quantifying hemozoin by optical reflectance spectrophotometry, directly on the patient's skin, on tissues or in a liquid sample which comprises means for calibrating the device; at least one optical emitter to excite the sample; at least eight optical detectors to detect the reflectance spectrum of the sample; at least eight bandpass optical filters to filter the reflected light for each optical detector; wherein the optical filters and optical detectors are aligned with each other, wherein the emitter and optical detectors are positioned allowing reflection of the emitted light towards the optical detectors, wherein the optical filters and optical detectors comprise wavelengths between 400 nm and 800 nm; and a microcontroller configured to calculate a ratio between the reflectance values of the sample at each wavelength to detect the reflectance peaks. Also disclosed is a method of detecting and/or quantifying hemozoin by optical reflectance spectrophotometry.

### TECHNICAL FIELD

The present disclosure relates to the field of diagnostic techniques, and relates to an automatic system for malaria detection, through contact with the patient skin or fluidic sample, and in a non-invasive way, based on the determination of the presence and quantification of hemozoin (Hz), by reflectance spectrophotometry, and respective detection methods.

### BACKGROUND

Malaria is a severe infectious disease caused by a protozoan parasite, and still considered a serious public health global problem. In 2018, 228 million cases were reported, 93% of them in Africa and with the highest incidence in children under 5 years old, leading to the annual death of more than 400,000 people. The estimate of these numbers is conservative, with the actual numbers being probably higher, considering that many of the cases are untested or not even reported. Thus, the ability to achieve an accurate diagnosis becomes a critical factor for the control and eventual elimination of malaria.

Current diagnosis relies on the detection of parasites in the blood through light microscopy and, more recently, through a panoply of rapid diagnostic tests (RDT) based on immunochromatography. Both present advantages and disadvantages. Optical microscopy allows the quantification and distinction between species, but requires microscopes and specialized and qualified technicians, which can lead to a subjective interpretation of results. It is a relatively inexpensive method but, due to the requirements described above, it does not allow its use in many endemic regions, especially in more remote locations. With a comparable sensitivity (detection limit around 50 parasites/μL of blood), RDT do not require such qualified personnel and, as a consequence, allow their use in more remote areas. However, RDT are expensive (often more expensive than the treatment itself) and do not allow for the quantification of parasites. Regarding sensitivity, neither of the methods has the high level of sensitivity that molecular diagnosis by PCR (Polymerase Chain Reaction) provides. However, the latter is not used as a routine diagnosis, as it is not feasible to be performed outside diagnostic laboratories. In addition, these techniques require the collection of a blood sample, as well as disposable reagents or consumables. In the current era, in the search for malaria control and eventual elimination of the disease, new diagnosis technologies are needed, being (a) simple, (b) fast, (c) of high sensitivity, (d) low cost, (e) minimally or non-invasive and (f) with more environmentally sustainable alternatives, for in-situ use, in endemic regions or for screening at the first symptoms of malaria, which do not require specialized technical personnel to perform the test, detecting the presence of the parasite in an early stage and allowing to monitor the disease therapy response.

In humans, symptoms of infection start to appear when malaria parasites (Plasmodium) enter the bloodstream in the form of merozoites, invading the red blood cells. Within the red blood cells, the parasites undergo a cyclic maturation process that varies, in time, in periods of 24 to 72 hours, within the 5 species of Plasmodium that infect humans. The cycle takes place in 24 hours in the case of P. Knowlesi, 48 hours in the case of P. falciparum, P. vivax and P. ovale, and 72 hours in the case of P. malariae. During the cycle, the parasite, in the form of a merozoite, grows and multiplies within the red blood cell into about 8 to 32 new merozoites, that pass through the maturation stages from the ring, trophozoite and schizont forms. At the end of the cycle (schizont form), the red blood cell ruptures, releasing new merozoites which, in turn, will infect more red blood cells. This cyclical process of parasite maturation leads to a set of biochemical and morphological changes in the red blood cells that, taking into account the cyclical and morphological temporal differences detected by microscopy, may also differ between species of the parasite. One of these changes is the degradation of hemoglobin (Hb), which is the main nutrient necessary for the parasite's metabolism during its intracellular development. The degradation of hemoglobin by the parasite, within its digestive vacuole, leads to the release of the prosthetic group heme, with toxic properties. The parasite detoxifies, forming crystalline particles of heme groups, denominated hemozoin (Hz), also known as the malaria pigment, which accumulates in the parasite's digestive vacuole as the intra red blood cell cycle proliferates, while the Hb concentration decreases. Since Hz is a unique particle of the parasite, healthy human blood does not have Hz. In patients infected with malaria, the concentration of Hz increases as the parasite biomass load increases, which causes the disease to progress. Thus, determining the amount of Hz in a sample can help to indicate the presence of the malaria parasite and its parasitemia. Additionally, the Hb and Hz molar extinction coefficients differ significantly, especially at certain wavelengths, which leads to different absorption and reflection optical spectra, of normal and infected red blood cells. Thus, it is possible to identify and quantify Hz by measuring the absorption and/or reflection spectra of blood (whole or fractionated blood), analyzing the peaks and variations of Hb and Hz, which will act as an important marker of malaria.

Previous studies 1. Silva et al., “Spectrophotometric Characterization of Hemozoin as a Malaria Biomarker”, Proc. SPIE 10453, Third International Conference on Ap-plications of Optics and Photonics, 1045304 (22 Aug. 2017); AOP2017, Faro, Portugal, 8-12 May 2017 (DOI: 10.1117/12.2270995) and I. Silva et al., “Hemozoin and hemoglobin characterization by optical absorption towards a miniaturized spectro-photometric malaria diagnostic system”, 5th IEEE Portuguese BioEngineering Meeting, Coimbra, Portugal, 16-18 Feb. 2017 (DOI: 10.1109/ENBENG.2017.7889466) disclose the existence of a Hz optical absorption peak at 672 nm, as well as the possibility of detecting small variations in the concentration of Hz in whole blood samples, using the visible area of the optical spectrum, and using reduced sample volumes. However, these studies use high-cost and commercial spectrophotometry equipment, located in a laboratory environment, therefore not portable, and are not based on non-invasive reflectance techniques.

The documents S. O. Catarino et al., “Portable device for optical quantification of hemozoin in diluted blood samples”, IEEE Transactions on Biomedical Engineering, 2019 (DOI: 10.1109/TBME.2019.2913454) and PPT 110564 describe an automatic and portable device to detect and quantify Hz in diluted whole blood samples, based on optical absorption measurements at specific wavelengths, detecting the Hz absorption peak present at 672 nm. However, the device presented in the documents is based on optical absorption spectrophotometry and involves the use of a blood sample, thus being invasive.

The document U.S. Pat. No. 7,236,236B2 describes methods for detecting the malaria parasite, which include releasing the malaria parasite, by hemolysis of the red blood cells (using a flow cytometer), labeling the parasite with a fluorescent markerto prepare a sample for measurement, detection of the optical information of the sample (fluorescence intensity emitted by the sample) and the detection of the parasite based on the obtained optical information. However, the device is not portable or non-invasive and needs fluorescent markers.

The document U.S. Pat. No. 8,920,726B2 describes a device for blood analysis and detection of malaria that includes the preparation of blood samples with a hemolytic agent, a coloring agent and a control portion, for measuring and classifying blood cells as being infected or not by malaria, based on fluorescence and light scattering signals.

The document U.S. Pat. No. 9,541,552B2 describes a device for analyzing a hemolyzed blood sample, which includes a light source at a wavelength for which the Hb in a hemolyzed blood sample has absorbance (in the green region of the spectrum, around 570 nm). Between the light source (broad-spectrum, which can be white light or light in the green range) and the sample where the light falls, there is a filter to remove the light outside the wavelengths' range of interest. The device includes a pixel detector surface with an array of photodetectors that quantifies the output light from the sample, in a wavelength band corresponding to at least a portion of the green light spectrum, and generates signals representative of the amount of Hb in the sample and of the amount of malaria parasite in the sample. Between the sample and the detector, there is a lens system to focus the optical path of the light to the surface of the detectors. Through a processor, an algorithm is applied to generate a red blood cells value, based on the Hb signal, and a parasitemia value (percentage of red blood cells infected by malaria parasites) based on both the parasite signal and the red blood cells value.

The document U.S. Pat. No. 9,046,473B2 describes a method and setup for determining intraerythrocytic organisms, in particular anisotropic crystals (such as Hz) in a liquid sample of whole blood. This method includes a light source (which illuminates the sample in the spectrum region between 410 and 420 nm), an optical detector to create an image of the sample and a processing program to analyze the obtained images. The images are then analyzed to detect the presence of, at least, one red blood cell in the image containing a region with a decreased amount of Hb and/or with a lower concentration of Hb in the red blood cell, determining the presence of the intraerythrocytic organisms in the sample. Additionally, a fluorescent label can be added to the sample to detect the presence of Hz based on the relative differences between the red blood cells fluorescence.

The document WO2009/009899A1 is based on the fact that Hz has a strong non-linear optical response, producing a third harmonic signal when excited by a laser optical signal, and describes a system for detecting Hz in a sample of blood, blood cells, tissues or other media. The system includes a light source (preferably an infrared laser) to excite the sample and generate a non-linear optical response, lenses or mirrors to focus the optical signal into the sample, a detector to detect the non-linear response signal from the excited sample (preferably a photomultiplier tube, but can also be a photodiode or photodiode array).

The document WO2016/066754A1 is based on the magnetic properties of Hz as an indicator of malaria infection. The presence of Hz in a whole blood (lysate) sample is detected in a process that involves: magnetic separation of the sample in the device, dissolution of the magnetic separated component to obtain a solution, comprising the target material, that can be analyzed, and spectroscopic analysis. The spectroscopic analysis includes optical absorption spectrometry, using quasi-monochromatic light, with wavelengths in the 350-420 nm or 600-640 nm range. The method allows to detect a Hz concentration under 0.1 μg/ml in the sample, preferably under 0.08 μg/ml. The cited patent document, by analyzing the optical behavior of Hz in regions of shorter wavelengths, implies a magnetic separation phase (applying magnetic fields) of the Hz from the lysed whole blood, to remove the effect of the blood absorption in these regions of the spectrum, as well as a phase of dissolution in an aqueous solution with an alkaline agent. The present invention uses the reflectance spectrum of blood and Hz, not implying blood samples.

Documents U.S. Pat. No. 8,388,509B2, U.S. Pat. No. 8,467,842B2 and U.S. Pat. No. 8,840,536B2 describe systems, devices and methods to, among other applications, detect Hz, as well as for diagnosing, monitoring or treating a malaria infection. The systems include sensors for detecting a non-linear multi-harmonic response associated with the Hz nanoparticles in a biological tissue subjected to an electromagnetic stimulus (with wavelength peaks between 690 nm and 2100 nm). The sensor is configured to detect a non-linear response using one or more differential illumination settings (dark field illumination, Rheinberg). The energy response associated with the Hz nanoparticles is compared to a reference response profile. The present invention uses non-invasive methods. Additionally, the cited documents detect the Hz non-linear scattering response, while the present invention measures the Hz reflectance. These documents use much more complex and costly methods than the device in the present disclosure, which uses the spectrophotometric analysis by optical reflection as the detection method.

The document U.S. Pat. No. 8,214,006B2 is based on the detection of Hz through the change in the magnetic state of hemoglobin, caused by the malaria infection. The properties of Hz vary with the application of a magnetic field, so a potentially non-invasive opto-acoustic detection technique based on the alteration of these properties is proposed. The experimental apparatus includes a light source, which produces a beam of optical radiation that is filtered and focused on a sample placed on a support, and which is in direct contact with an acoustic detector. The apparatus also includes an electromagnet and a gaussimeter to measure the strength of the applied magnetic field, allowing for in vivo measurements. The present invention differs from the referred document, since it is independent of the magnetic properties of Hz, so it does not imply the application and measurement of magnetic fields.

Documents E. Y. Lukianova-HIeb et al., “Hemozoin-generated vapor nanobubbles for transdermal reagent- and needle-free detection of malaria”, PNAS, 2014 (DOI: 10.1073/pnas.1316253111) and E. Y. Lukianova-Hleb et al., “Transdermal Diagnosis of Malaria Using Vapor Nanobubbles”, Emerging Infectious Diseases, 2015 (DOI: 10.3201/eid2107.150089) present a detection method based on hemozoin-induced vapor nanobubbles (H-VNBs), which work as optical and acoustic probes of high sensitivity for the detection of malaria. The application of a laser pulse in the infrared range leads to heat release in nanovolumes around the hemozoin nanoparticles and consequent liquid evaporation, leading to the formation and collapse of vapor nanobubbles around the particles. Then, the presence of nanobubbles is detected based on the generated acoustic signals.

These facts are disclosed in order to illustrate the technical problem addressed by the present disclosure.

### GENERAL DESCRIPTION

The present disclosure comprises a device and method for non-invasive detection of Hz, based on optical reflectance spectrophotometry, capable of detecting Hz at low parasitemia, namely from 12.5 parasites/μL (sensitivity better than the current diagnostic methods), enabling the detection directly on the patient's skin or, alternatively, in contact with other tissues, such as the tongue. Optionally, fluidic blood samples can be used in the device for the detection of Hz. With this device, when used on the patient's skin, it is not necessary to take blood samples, it is non-invasive, there is no need for control samples, it is simple and fast, eliminates the subjectivity associated with the results interpretation by the technicians, it can be used in remote areas and is ecological, since it eliminates the need for disposable reagents and cartridges and, as it is non-invasive, it does not produce residues resulting from sample collection, such as needles, cotton, gloves or band aids.

The present disclosure is distinguished from other approaches by identifying, in-situ and without the need to collect blood samples, the presence and the quantity of Hz through optical reflectance spectrophotometry, with the aim of detecting the presence of malaria parasites and the parasitemia, using specific wavelengths in the reflectance spectra of whole blood and hemozoin as an important marker of this disease. In addition, the device, according to the present invention, comprises a reference measurement system, essential not only for the correct measurements, but also ensuring that the decrease in the illumination capacity of the light source, due to the aging of the electronic components over their lifetime, is negligible. This reference measurement also allows the device not to be affected by temperature variations that could change the performance of the electronic components. When compared to the conventional methods, this disclosure is distinguished by not requiring the collection of any type of sample, namely blood. Particularly, when compared to RDT, as this disclosure allows to detect the relative intensity of Hz peaks in comparison to healthy blood spectra, it has the advantage of helping to quantify the parasites. Additionally, benefiting from the cyclic and morphological differences between the different species of the parasite, this disclosure may allow the distinction between species based on their spectra. Furthermore, taking advantage of on-chip technologies, this method, when compared to microscopy, allows to detect the infection without the need for specialized technicians, reducing the error rate due to subjectivity, and producing quick results. When compared to PCR, detection by reflectance spectrophotometry allows the results to be obtained without the need for collecting samples. When compared to other optical methods, the proposed solution has distinctive technical characteristics, with the advantage of involving less instrumentation, reducing the cost and the complexity of the device, and providing portability.

In an embodiment, the device combines an optical emission system, comprised of white light, or alternatively Light Emitting Diodes (LEDs) or laser diodes and actuation electronics; an optical detection system, comprising optical filters and photodetectors, reading electronics and a microcontroller; and a power supply system.

In an embodiment, the operating principle is based on the optical reflectance detection of Hz and on an algorithm that correlates its values between different wavelengths of the spectrum. The light emitted by the device is sent to the tissue to be analyzed like patient's skin or other tissue such as the tongue or in a liquid sample. Part of the incident light is reflected and the intensity of this light, at specific wavelengths, which is indicative of the concentration of the biomolecule under analysis, is filtered using optical bandpass filters at different wavelengths of the optical spectrum, and is measured by a set of photodetectors placed close to the emitting source (and properly isolated from it). An algorithm relates the normalized reflectance values between the several considered wavelengths. The variation in the normalized reflectance values between the various wavelengths, and the different quotients between them are indicative of the presence of Hz in the sample.

In an embodiment, the portable device for detecting and/or quantifying hemozoin by optical reflectance spectrophotometry directly on the patient's skin, tissues or a liquid sample comprises:
optical reflectance spectrophotometry directly on the patient's skin, tissue or a liquid sample comprising
means for calibration of the device or calibration means;
at least one optical emitter to excite the sample;
at least eight optical detectors for detecting the spectral reflectance directly on the patient's skin, tissues or a liquid sample;
at least eight bandpass optical filters to filter the reflected light for each optical detector;
wherein the optical filters and optical detectors are aligned with each other;
wherein the emitter and detectors are positioned allowing the reflection of the emitted light towards the optical detectors;
wherein the optical filters and optical detectors comprise wavelengths between about 400 nm to 800 nm;
and a microcontroller configured to calculate the ratio between the reflectance values of the sample at each wavelength, for detecting the reflectance peaks, for detecting and quantifying hemozoin.

In an embodiment, the variations in the normalized reflectance values between the several wavelengths, and the different quotients between them, are indicative of the presence of Hz in the sample.

In an embodiment, the sample is an in vitro or an in vivo.

In an embodiment, the optical emitter is a white light source, LEDs, laser diodes or combinations thereof.

In an embodiment, the device comprises at least 8 independent spectrophotometry emitters, when the optical emitters are LEDs or laser diodes.

In an embodiment, the device comprises at least 8 optical emitters, preferably 8, 9, 10, 11, 12, 13, 14, 15, 16 independent emitters.

In an embodiment, the emitters have a wavelength between around 400 nm and 800 nm.

In an embodiment, the device comprises 9, 10, 11, 12, 13, 14, 15, 16 optical detectors and respective filters.

In an embodiment, the means for calibration of the optical device/system include the measurement of the reflectance values of a reference or standard sample.

In an embodiment, the reference or standard sample is a barium sulphate sample.

In an embodiment, the means for placing the reference sample for calibration is a support.

In an embodiment, the device comprises means for contacting the sample.

In an embodiment, the device comprises a window configured to be in contact with the patient's skin.

In an embodiment, the emitters are configured to emit light at a specific wavelength.

In an embodiment, the LEDs and laser diode emitters or combinations thereof emit at a wavelength range around: 400 nm, 435 nm, 520 nm, 590 nm, 610 nm, 620 nm, 630 nm, 640 nm, 650 nm, 660 nm, 670 nm, 680 nm, 700 nm, 720 nm, 740 nm, 800 nm.

In an embodiment, the device also comprises a power supply.

In an embodiment, the power supply is a cell, a battery or combinations thereof.

In an embodiment, the sample to be analyzed is skin, tissue or a liquid biological sample.

In an embodiment, the device measures the reflectance directly on the patient's skin or other tissues, such as the tongue.

In an embodiment, the wavelength of the first emitter is about 400 nm, the wavelength of the second emitter is about 435 nm, the wavelength of the third emitter is about 520 nm, the wavelength of the fourth emitter is about 590 nm, the wavelength of the fifth emitter is about 610 nm, the wavelength of the sixth emitter is about 620 nm, the wavelength of the seventh emitter is about 630 nm, the wavelength of the eighth emitter is about 640 nm.

Another aspect of the present disclosure describes a method for detecting and/or quantifying hemozoin by optical reflectance spectrophotometry directly on the patient's skin, tissue or liquid sample comprising the following steps:
determining the reflectance of a barium sulphate reference sample;
determining the reflectance of the sample to be analyzed after the emission of an optical beam;
and calculating the discrete reflectance of the sample at each wavelength;
calculating the normalized reflectance of the sample at each wavelength; and
calculating the ratios between the normalized reflectance values at each wavelength for detecting the discrete reflectance slopes of the different wavelengths or calculate the area under the spectrum of the normalized reflectance.

In an embodiment, the device can comprise a white light source (or, instead, a set of LEDs or laser diodes) and a detection set with 16 bandpass optical filters and an equal number of photodetectors (for example photodiodes), in number equal to the number of relevant wavelengths, and encapsulated in order to ensure the optical isolation between the emission and detection systems, so the device should have a dark color to prevent the entry of external light into the device. In a different configuration, the system can contain a smaller number of wavelengths and photodetectors, as long as they fall between the 400 nm and 800 nm regions.

In an embodiment, the white light source emits light across the entire visible spectrum. In other configurations, LEDs or laser diodes emit light at a range of specific wavelengths, between 400 and 800 nm, for example around the regions: 400 nm, 435 nm, 520 nm, 590 nm, 610 nm, 620 nm, 630 nm, 640 nm, 650 nm, 660 nm, 670 nm, 680 nm, 700 nm, 720 nm, 740 nm, 800 nm, in order to gather information from different regions of the visible area of the optical spectrum, for the construction of a decision algorithm that allows the distinction between the presence or absence of Hz reflectance peaks. The choice of the wavelength range of interest was made after carrying out experimental tests with a commercial spectrophotometric system, and took into account the optical properties of blood, in particular the reflectance peaks of red blood cells and whole blood, the changes of the optical properties of whole blood with the presence of Hz (FIG. 1), as well as the effect of the skin and other tissues. However, in other configurations, different wavelengths in these spectral ranges can be used, always considering reflectance signals in the visible region of the optical spectrum.

Thus, at each wavelength, the light intensity detected by the photodiode is dependent on the characteristics of the sample: hematocrit, tissues and the presence and amount of Hz. The use of a normalized reflectance curve reduces the influence of the hematocrit in the reflectance, allowing to detect the presence of Hz.

In an embodiment, the light beams will focus on the skin of the patient or sample to be analyzed and will be reflected. The reflected light will be filtered through a set of optical bandpass filters, optimized for the wavelengths of interest, and will be detected by the photodiodes. The system is controlled by a microcontroller and current-voltage converters, in order to produce a voltage value proportional to the current generated by the photodiodes, and which can be acquired by the ADC (Analog to Digital Converter) of the microcontroller.

In an embodiment, a reference measurement system guarantees, not only the correct measurement, but also that the decrease in the lighting capacity due to the aging of the electronic components over the respective lifetime are negligible. This reference also allows the device not to be affected by temperature variations that could alter the performance of the electronic components.

In an embodiment, the microcontroller receives the voltage values for each of the wavelengths (selected through the bandpass filters) and, based on the reference values (that is, values obtained only with a high reflectance sample of barium sulphate) it calculates the discrete reflectance values and normalizes them (relatively to the first wavelength of the spectrum). The normalization of the data allows to reduce, for example, the effect of hematocrit, better showing the variations and slopes between the wavelengths resulting from the presence of Hz. After calculating the normalized reflectance values, the microcontroller executes a decision algorithm in order to classify the sample as containing or not Hz (thus indicating the presence or absence of the malaria parasites). In the presence of parasites, there is a greater difference in the slopes between the normalized reflectance values at various wavelengths, which is as higher as the parasitemia increases, so the algorithm is based on the variation between the reflectance values at the analyzed wavelengths.

More specifically, in an embodiment, after calculating the reflectance of the sample at the selected wavelengths (e.g. 400 nm, 435 nm, 520 nm, 590 nm, 610 nm, 620 nm, 630 nm, 640 nm, 650 nm, 660 nm, 670 nm, 680 nm, 700 nm, 720 nm, 740 nm, 800 nm), the following step sequence of the algorithm is performed:
1) normalization of the reflectance spectrum around a unit value for the first considered wavelength (in this case, 400 nm), applying the same correction factor to the reflectance of the same sample at the other wavelengths.
2) calculation of the slopes between the sample reflectance values at the various wavelengths in the range of 400 nm to 800 nm, to aid in the reconstruction of the optical spectrum.
3) a positive slope, that is, an increase in reflectance between wavelengths, represents the rise to a peak or region of high reflectance, while a negative slope indicates the relative decrease of the reflectance between the wavelengths under analysis.
4) for example, in the realization observed in FIG. 6, whose slopes were determined by experimental tests, the presence of Hz is detected by an increase in slopes, above 0.015, between the wavelengths of 583 nm and 606 nm (or close ranges) in the reflectance spectra, and a decrease in the absolute values of the slopes between 606 nm and 651 nm, below 0.001 (in absolute value). The differences and slopes between the different wavelengths represent the variation in the optical reflectance spectrum resulting from tissues, hemoglobin (which is lower according to the more advanced the malaria stage), helping to reconstruct the optical spectra and detect the presence of Hz. The device and the sample classification algorithm, in particular the threshold slope values for identifying Hz in the sample must be calibrated, since the threshold slope values to be used also depend on the technical characteristics of the optical filters, in particular their transmittance and their width at half height, which can vary significantly with the device configuration. These differences and the slopes and limits for comparison were previously determined through experimental tests.

In an embodiment, as an alternative to calculating slopes, the detection algorithm is based on calculating the area under the normalized reflectance spectrum of the samples, which is higher as the greater the amount of Hz in the tissue or sample.

In an embodiment, once the data is read, compared and the decision algorithm is executed, the system will send the information to the display, communicating with the user. The result of the test is displayed on the microcontroller's screen, thus providing the user with real-time information. The screen can be tactile or not, and test information (user and test result) can be stored on a memory card and/or transmitted to a computer via serial communication or via a wireless system, or via USB, among others.

Furthermore, the algorithm is based on measuring the optical reflectance values in a wider spectral range, considering the correlation between the different spectral values, which allows for better reliability in the quantification of the parasitemia.

An aspect of the present embodiment describes a portable device for detecting and/or quantifying hemozoin by optical reflectance spectrophotometry directly on the patient's skin comprising one or more independent spectrophotometry emitters to excite each sample, where they emit white light or, alternatively, where they emit at the various relevant wavelengths between 400 nm and 800 nm;
at least eight optical detectors with bandpass optical filters and photodetectors to detect reflectance at each wavelength;
where the emitters and detectors are positioned so as to guarantee the reception of light after it is reflected;
means for the system reference calibration,
and a microcontroller capable of calculating the relation between the reflectance at each wavelength, in order to detect the reflectance peak.

In an embodiment, the device may comprise an additional emitter and optical detector thereof.

In an embodiment, the device can comprise up to sixteen relevant wavelengths, with the respective optical detectors.

In an embodiment, the device comprises means for calibration of the device from a reference measurement.

In an embodiment, the optical emitters are white light regular sources.

In an embodiment, alternatively, the optical emitters are LEDs, laser diodes, or combinations thereof.

In an embodiment, each LED, laser diode or combinations thereof emits at a specific range of wavelengths.

In an embodiment, each LED, laser diode, or combinations thereof emits at a wavelength range around: 400 nm, 435 nm, 520 nm, 590 nm, 610 nm, 620 nm, 630 nm, 640 nm, 650 nm, 660 nm, 670 nm, 680 nm, 700 nm, 720 nm, 740 nm, 800 nm or other combinations.

In an embodiment, the device comprises bandpass optical filters for each of the wavelengths of interest, positioned over the photodiodes.

In an embodiment, the device also comprises a power supply. Preferably, the power supply is a cell, battery or combinations thereof.

In an embodiment, the device does not require a sample, measuring the reflectance directly in contact with the patient's skin.

The present disclosure further relates to a method for detecting and/or quantifying hemozoin by optical reflectance spectrophotometry in a patient comprising the following steps
determine the reflectance of a reference sample of barium sulphate;
determine the reflectance of a sample at:
   a minimum of eight wavelengths between 400 and 800 nm;
calculate the discrete reflectance of the sample at each wavelength;
calculate the normalized reflectance of the sample at each wavelength;
calculate the relation between the normalized reflectance values at different wavelengths, in order to detect a variation in the slopes of the reflectance spectrum due to Hz.

In an embodiment, as an alternative to analyzing the sample based on the slopes between the wavelengths, the method of detection and/or quantification of hemozoin by optical reflectance spectrophotometry can be performed by calculating the area under the normalized reflectance spectrum reconstructed at the relevant wavelengths.

In an embodiment, the present device allows the detection of Hz in the patient's skin or other samples through the variation of the reflectance values of the samples up to sixteen wavelengths. The method does not involve processing of samples or reagents.

In an embodiment, the device is portable, comprised of one or more PCB boards (Printed Circuit Board) with emission electronics, with the set of emitting sources and electronic circuits to control which emitting source is operating in each moment, and with photodiodes and current-to-voltage converters.

In and embodiment, as an alternative to the PCB boards, a CMOS (Complementary Metal-Oxide-Semiconductor) chip can be used.

In an embodiment, other configurations are also possible. The chosen configuration needs to ensure the light reflection between the emitter beam, the patient's skin (or other test surface) and the photodiodes for acquisition of the reflected light. The device can be used in any environment, as it is not affected by external light. This feature is due to the construction of the device in black color, designed to ensure total isolation from ambient light.

### BRIEF DESCRIPTION OF THE DRAWINGS

For an easier understanding of the present disclosure, the following figures are attached, which represent preferred embodiments which, however, do not intend to limit the object of the present disclosure.

FIG. 1 represents four normalized optical reflectance curves, measured on commercial spectrophotometric equipment, including a normalized reflectance spectrum of healthy red blood cells (RBC) in the visible region of the optical spectrum, and reflectance spectra of RBC with Plasmodium falciparum parasites, at the trophozoite stage, with parasitemia of 12.5 parasites/μL, 25 parasites/μL and 50 parasites/μL.

FIG. 2 represents four normalized reflectance analyses, with spectra reconstructed from sixteen wavelengths within the proposed range, measured on a commercial spectrophotometer, on a healthy red blood cells (RBC) sample and three RBC samples with Plasmodium falciparum parasites, at the trophozoite stage, with parasitemia of 12.5 parasites/μL, 25 parasites/μL and 50 parasites/μL.

FIG. 3 shows an example of a set of slopes calculated between the normalized reflectance at various wavelengths, from the reconstructed spectra of FIG. 2, in the absence of filters and measured in a commercial spectrophotometer, where it is visible the difference between healthy samples and with parasitemia.

FIG. 4 represents an example of a set of eight bandpass optical filters, for eight wavelengths, and the respective optical transmittance spectra. The differences in the transmittance and bandwidth of the different bandpass filters have a direct influence on the resulting spectra, as seen in FIG. 5.

FIG. 5 represents the normalized reflectance spectra, measured with the device, obtained from the measurements of the electric current at the photodiodes under the eight considered bandpass filters (FIG. 4) and respective wavelengths of interest, for a healthy red blood cell (RBC) sample and three RBC samples with Plasmodium falciparum parasites, at the trophozoite stage, with parasitemia of 12.5 parasites/μL, 25 parasites/μL and 50 parasites/μL.

FIG. 6 represents an example of a set of slopes calculated between the normalized reflectance at different wavelengths, measured with the device, obtained from the measurements of the electric current at the photodiodes under the eight considered bandpass filters and their respective wavelengths of interest, for a sample of healthy red blood cells (RBC) and three blood samples with parasites at the trophozoite stage with parasitemia of 12.5 parasites/μL, 25 parasites/μL and 50 parasites/μL.

FIG. 7 represents examples of the area under the normalized optical reflectance spectra at different wavelengths of interest, for healthy blood samples and blood samples with parasites at the trophozoite stage, with parasitemia of 12.5 parasites/μL, 25 parasites/μL and 50 parasites/μL. The plot shows the areas for the full normalized reflectance spectra, measured on a commercial spectrophotometer, for the normalized reflectance spectra reconstructed from 16 discrete wavelengths, also measured on a commercial spectrophotometer, as well as for the normalized reflectance spectra measured with an embodiment of the device, with 8 optical filters (FIG. 4) and respective wavelengths.

FIG. 8 represents an embodiment of the device, in an upper view, in which 1 corresponds to the packaging, in black color for a better light isolation between the various components, 2 the button to turn the system on and off, 3 the screen for presenting the results, 4 the memory card input, 5 the measurement area, with a support for measuring the barium sulphate reference sample, together with the lighting and optical detection systems.

FIG. 9 represents an example of a view of the electronic components of the device, in which 6 corresponds to the lighting system, comprising a white light source, LEDs or Laser diodes, and 7 to the optical detection system, consisting of a photodiode array and optical filters (positioned and aligned on top of the photodiodes).

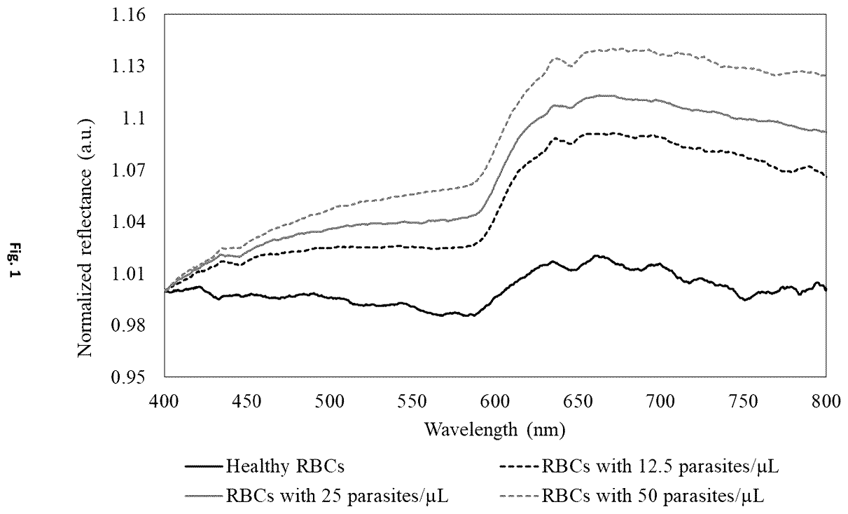

### DETAILED DESCRIPTION

The present disclosure presents a portable device and method for detecting, non-invasively, the presence of malaria parasites and their quantification, by optical reflectance. The device combines an optical emission system, comprised of white light, or, instead, LEDs or laser diodes for emission of the light beams, and electronics for their actuation, an optical detection system, comprised by bandpass optical filters and photodetectors, a microcontroller, the reading electronics, which consists of current-voltage converters, in order to produce a voltage value proportional to the current generated by the photodetectors, and which can be acquired by the ADC of the microcontroller, and the power supply system, being optically isolated from the outside to prevent light from entering the system.

In an embodiment, the device comprises optoelectronic components, including a white light source that emits throughout the visible spectrum, or alternatively a set of LEDs or Laser diodes, which emit at specific wavelengths between 400 nm and 800 nm, preferably at: 400 nm, 435 nm, 520 nm, 590 nm, 610 nm, 620 nm, 630 nm, 640 nm, 650 nm, 660 nm, 670 nm, 680 nm, 700 nm, 720 nm, 740 nm, 800 nm or other combinations, in order to gather information from different regions of the visible area of the optical spectrum, for the construction of a decision algorithm that allows the distinction between the presence or absence of Hz reflectance peaks, as well as electronic control circuits with Pulse Width Modulation (PWM) control.

In an embodiment, different wavelengths in the visible range can be used, always on the 400 nm to 800 nm spectral range.

In an embodiment, the device also comprises up to sixteen optical filters centered on the wavelengths of interest (in case a white light source is used), and the same number of photodiodes (or other photodetectors), placed close to the emitting source (properly insulated), aligned with each other, and current-voltage converters; a microcontroller for controlling the optoelectronic components and for performing the analysis and interpretation of the values obtained by the photodiodes; a display for viewing the test result.

In an embodiment, the device comprises a lock-in amplifier that amplifies the collected low-amplitude signals and eliminates their noise.

In an embodiment, the optical filters and the photodiodes are aligned with each other and spaced apart from each other (in the horizontal direction) and encapsulated so as to ensure optical isolation of the emission and detection systems from external light entering the system.

In an embodiment, the system may contain a smaller number of optical filters and photodiodes, as long as it contains at least eight, between the 400 nm and 800 nm optical regions.

The present example of the use of the disclosure is demonstrative, not intending to limit the scope of protection.

In an embodiment, a reference sample is measured before each analysis, which allows the analysis to be carried out correctly, regardless of changes in the materials or ambient light, standardizing the optical measurements.

In an embodiment, the first step of the method for detecting the presence of Hz as a marker for the presence of malaria parasites, consists in obtaining the reference sample (with a barium sulphate sample, considered an international reference with 99.8% reflectance).

In an embodiment, for best results, a set of reference values is obtained before each analysis, or when the analysis conditions are changed, allowing the analysis to be performed correctly, regardless of changes in the material or environment, calibrating and standardizing the optical measurements:
place a disk with a barium sulphate reference sample in a specific socket in the device, with the test region in direct contact with the optical emission and detection systems, and activate the light emission system, which, after being reflected by the sample, will pass through the different optical filters and will be received by the photodiodes, being converted into a voltage value, which will be read by the microcontroller.
control the intensity of the light emission system through a pulse width modulated signal (PWM).
store the reference voltage values of each photodiode for later calculation of the optical reflectance.

Then, in an embodiment, the analysis of the sample's reflectance is carried out, following the steps:
the sample, which consists of the patient's skin, other tissues or a fluid sample where the presence of Hz is to be detected, is placed against a specific region of the device, with the test region being in contact with the optical emission and detection systems;
the system is once more activated and the white light source, LEDs or laser diodes emit a light beam that, upon reaching the sample, is partially reflected and directed to optical filters, optimized for the wavelengths of interest, and subsequently detected by photodiodes;
each photodiode captures the light intensity that is reflected at specific wavelengths and generates an electrical current which is proportional to the amount of light received (the value of which depends on the characteristics of the sample: tissues, hematocrit and the presence and amount of Hz) and is converted in a voltage value by the current-voltage conversion block;
the microcontroller calculates the sample's discrete reflectance values at each of the wavelengths of interest, through the ratio between the sample voltage and the reference voltage, measured with barium sulphate and at the same wavelength;
the microcontroller calculates the sample's normalized reflectance values at each of the wavelengths of interest, by dividing the reflectance value at the first considered wavelength (eg 400 nm) by itself so that, at this wavelength, the normalized reflectance presents the value one, and applying the same correction factor to the sample's reflectance values at the other wavelengths;
the microcontroller performs the sample classification;
in particular, in one embodiment, the microcontroller performs the classification of the samples using an algorithm: the slopes between the normalized reflectance values at the different wavelengths are determined in order to determine the presence of regions of higher and lower reflectance. If the calculation of the quotients and respective slopes indicates the presence of an increase in the normalized reflectance slope, namely between 583 and 606 nm, and above the limits considered normal (above 0.015, experimentally determined), and between the 606 and 651, a slope minor than 0.001 (in absolute value), the sample is classified as containing parasites, otherwise then no malaria parasites are present. In each embodiment, the sample classification algorithm must be calibrated through experimental tests, in particular the slope threshold values for identifying Hz in the sample, since the determined values will depend on the characteristics of the optical filters considered, in particular their transmittance and their full width at half height;
the results are visualized on the device's display, or stored on a memory card and/or transmitted to a computer by serial communication or via a wireless system.

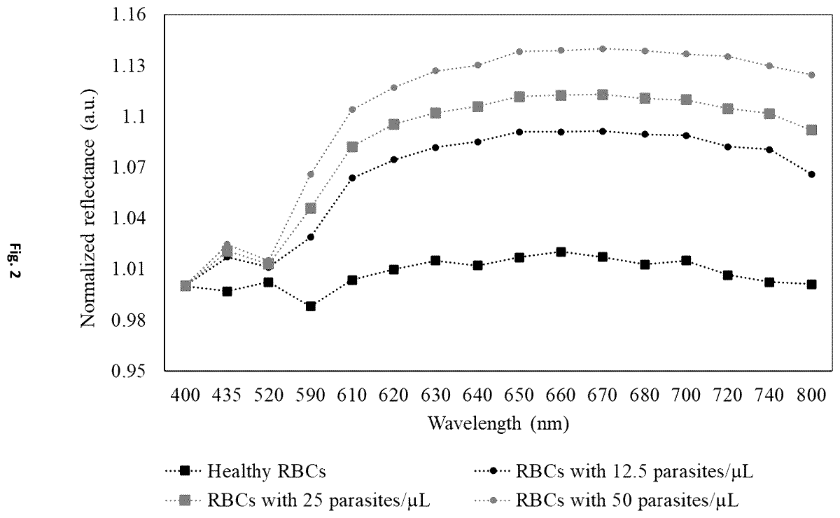

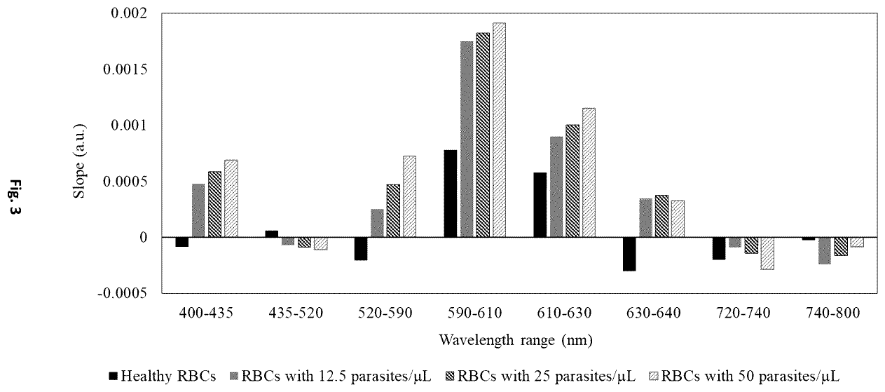

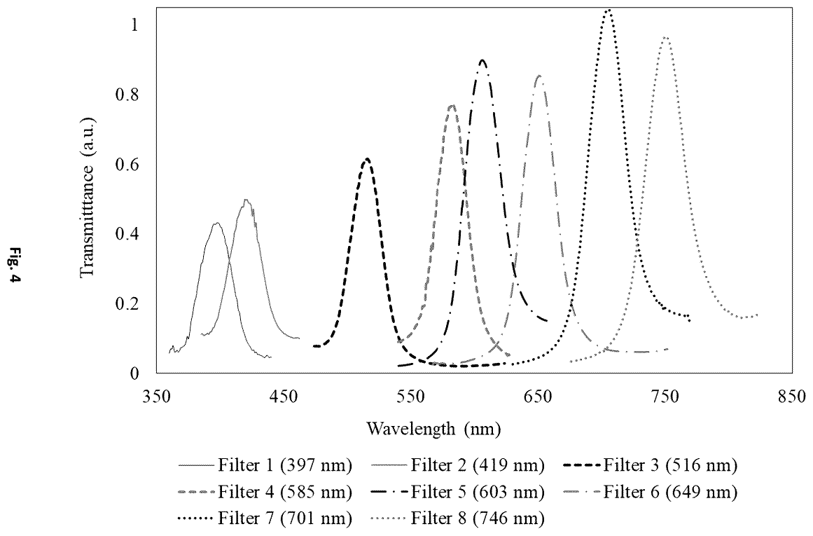

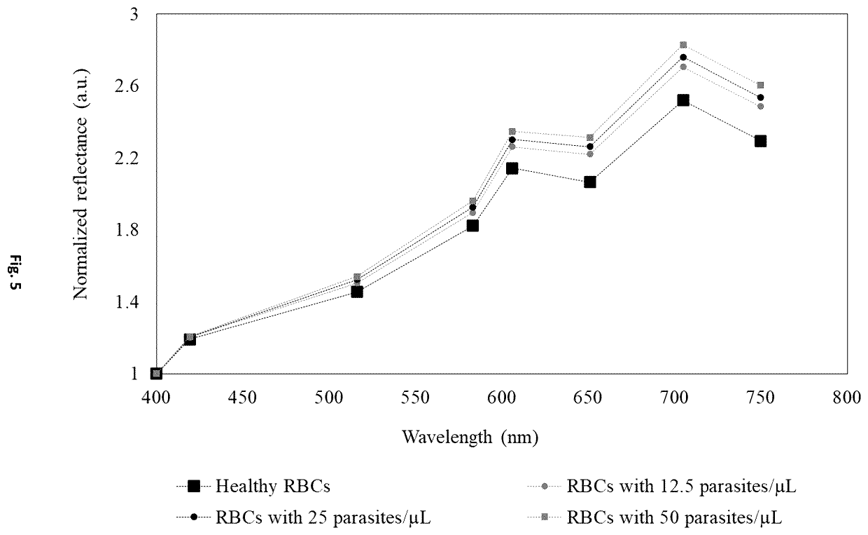

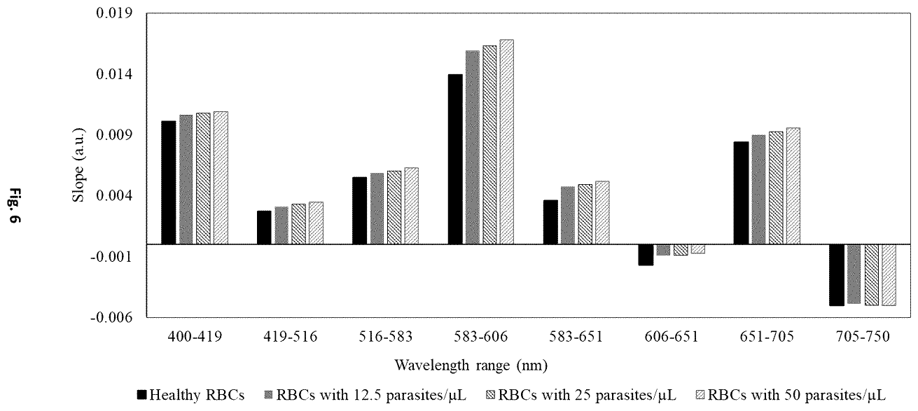

In an embodiment, FIG. 2 presents a normalized reflectance spectrum, constructed from 16 wavelengths, obtained for various RBCs samples with and without parasites, at the selected wavelengths. As can be seen, samples with Hz (parasitemia of 12.5 parasites/μL, 25 parasites/μL and 50 parasites/μL) have a higher amplitude of normalized reflectance above 600 nm, which is more significant as the concentration of Hz in the sample increases, as well as higher slopes between 510 nm and 610 nm, when compared to the sample of healthy RBCs, without parasites. FIG. 3 shows the slopes obtained between the different wavelengths of interest for healthy samples and samples with parasites, based on the reconstructed spectra shown in FIG. 2. FIG. 4 shows, as an example, the transmittance spectra of a set of optical bandpass filters to be used in the system and FIG. 5 shows the reconstructed reflectance spectra, obtained from the eight selected wavelengths (filtered by that set of optical filters), for several samples, with and without parasites. FIG. 6 presents a set of slopes calculated from the spectra shown in FIG. 5, from which it is possible to implement the sample classification algorithm. In the embodiment shown in FIG. 6, in the presence of a normalized reflectance slope greater than 0.015 between 583 and 606 nm, a slope greater than 0.0045 between 583 nm and 651 nm, and a slope minor than 0.001 (in absolute value) between 606 nm and 651 nm, the sample is classified as containing parasites, otherwise then no malaria parasites are present. The slope threshold values for malaria classification are experimentally determined. It is important to note that, when manufacturing each embodiment of the device, the device and the sample classification algorithm, in particular the slope threshold for identifying Hz in the sample must be calibrated, since the determined values will depend on the optical filters considered, in particular their transmittance and their full width at half height.

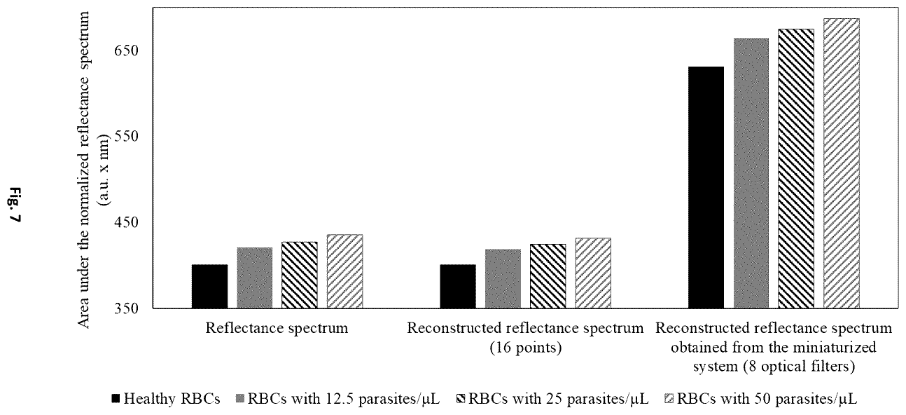

In an embodiment, as an alternative to classifying the sample based on the slopes between wavelengths, the method of detection and/or quantification of hemozoin by optical reflectance spectrophotometry can be performed by calculating the area under the reconstructed normalized reflectance spectrum (FIG. 7). In this embodiment, the device and the detection algorithm must be calibrated, after manufacturing, since the determined threshold areas will also depend on the characteristics of the optical filters considered, in particular their transmittance and their full width at half height.

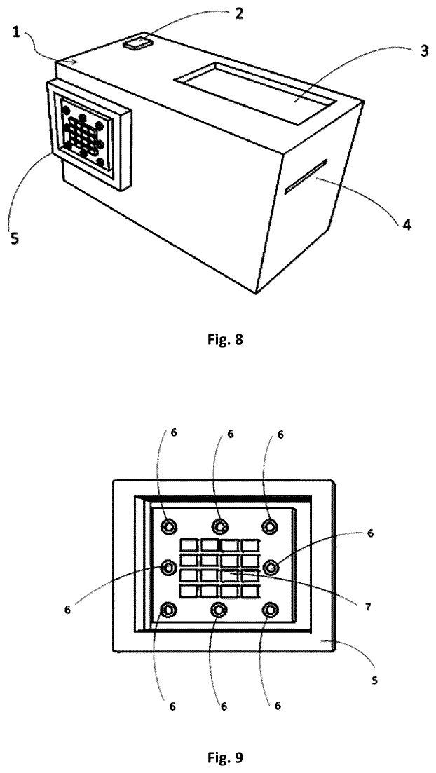

In an embodiment, FIG. 8 represents an example of the design of a final device, with the dimensions being adjustable, comprising the fitting to support the reference disc, ensuring its alignment with the optoelectronic components (FIG. 9), the emission and optical detection systems and the display for presenting the results. At the top of the device, there is the microcontroller, coupled to the display, with a space available for the system power supply. The packaging must be black in color and have no light inlets to ensure optical isolation, and to ensure that external light does not affect the measurements of the different photodetectors.

Throughout the description and claims, the term “comprises” and variations thereof are not intended to exclude other technical features, such as other components, or steps. Additional objects, advantages and features of the invention will become apparent to those skilled in the art upon examination of the description or may be learned by practicing the invention.

The embodiments and figures are provided by way of illustration, and are not intended to be limiting of the present disclosure. Furthermore, the present disclosure encompasses all possible combinations of particular or preferred embodiments described herein.

Although the present disclosure has only represented and described particular embodiments thereof, a person with ordinary skill in the art will foresee possibilities for modifications and replace some technical characteristics with equivalent ones, depending on the requirements of each situation, without leaving the scope of protection defined by the appended claims.

The above described embodiments are combinable.

The following claims further set out particular embodiments of the disclosure.

1. Portable device for detecting and/or quantifying of hemozoin by optical reflectance spectrophotometry directly on the patient's skin, tissues or a liquid biological sample comprising
means for calibration of the portable device from a reference measurement;
at least one optical emitter to excite the sample;
at least eight optical detectors for detecting spectral reflectance values directly on the patient's skin, tissues or a liquid sample;
at least eight bandpass optical filters to filter the reflected light for each optical detector;
wherein the optical filters and optical detectors are aligned with each other,
wherein the emitter and detectors are positioned allowing the reflection of the emitted light towards the optical detectors,
wherein the optical filters and optical detectors comprise wavelengths between about 400 nm to 800 nm; and
a microcontroller configured to calculate the ratio of the sample's reflectance value at each wavelength for detecting the reflectance peaks to detect and quantify hemozoin,
wherein it comprises at least eight independent spectrophotometry emitters when the optical emitters are LEDs or laser diodes, and
wherein the optical emitters have a wavelength between about 400 nm and about 800 nm.

2. The portable device according to claim 1, wherein the optical emitter is a white light source, LEDs, laser diodes or combinations thereof.

3. (canceled)

4. The portable device according to claim 1, wherein said device comprises at least 8 optical emitters.

5. (canceled)

6. The portable device according to claim 1, wherein said device comprises between 9 and 16 optical detectors and respective filters.

7. The portable device according to claim 1, wherein the calibration means of the optical device comprise the measurement of the reflectance values of a reference or standard sample.

8. The portable device according to claim 7, wherein the reference or standard sample is a barium sulphate sample.

9. The portable device according to claim 7, wherein the reference or standard sample is placed in a support.

10. The portable device according to claim 1, comprising means for contacting the sample.

11. The portable device according to claim 1, comprising a window configured to be in contact with the patient's skin.

12. The portable device according to claim 1, wherein the optical emitters are configured to emit light at a specific wavelength.

13. The portable device according to claim 1, wherein the LED emitters and laser diodes or combinations thereof each emit at wavelengths selected from the group consisting of about: 400 nm, 435 nm, 520 nm, 590 nm, 610 nm, 620 nm, 630 nm, 640 nm, 650 nm, 660 nm, 670 nm, 680 nm, 700 nm, 720 nm, 740 nm, and 800 nm.

14. The portable device according to claim 1, further comprising a power supply.

15. The portable device according to claim 14, wherein the power supply is a cell, a battery or combinations thereof.

16. The portable device according to claim 1, wherein the portable device measures the reflectance directly on the patient's skin or tongue.

17. The portable device according to claim 1, wherein:
the wavelength of the first emitter is about 400 nm,
the wavelength of the second emitter is about 435 nm,
the wavelength of the third emitter is about 520 nm,
the wavelength of the fourth emitter is about 590 nm,
the wavelength of the fifth emitter is about 610 nm,
the wavelength of the sixth emitter is about 620 nm,
the wavelength of the seventh emitter is about 630 nm, and
the wavelength of the eighth emitter is about 640 nm.

18. A method for detecting and/or quantifying hemozoin by optical reflectance spectrophotometry directly on the patient's skin, tissues or a liquid sample comprising the following steps:
determining the reflectance of a barium sulphate reference sample;
determining the reflectance of the sample to be analyzed after the emission of an optical beam by an optical emitter;
calculating the sample's discrete reflectance at each wavelength of the optical beam;
calculating the sample's normalized reflectance at each wavelength of the optical beam; and
calculating the ratio between the normalized reflectance values at each wavelength for detecting the discrete reflectance slopes of the different wavelengths or calculate the area under the spectrum of the normalized reflectance.
